(19) United States
(12) Patent Application Publication
(10) Pub. No.: US 2022/0336102 A1
(43) Pub. Date: Oct. 20, 2022

(54) LEARNING DEVICE, LEARNING METHOD, AND MEASUREMENT DEVICE

(71) Applicant: KABUSHIKI KAISHA TOKAI RIKA DENKI SEISAKUSHO, Aichi (JP)

(72) Inventors: Tetsuya HIROTA, Aichi (JP); Daisuke KAWAMURA, Aichi (JP); Yuuki NAWA, Aichi (JP); Minoru OTAKE, Aichi (JP); Ryugo FUJITA, Aichi (JP)

(73) Assignee: KABUSHIKI KAISHA TOKAI RIKA DENKI SEISAKUSHO, Aichi (JP)

(21) Appl. No.: 17/763,479

(22) PCT Filed: Aug. 11, 2020
(86) PCT No.: PCT/JP2020/030608

§ 371 Date: Mar. 24, 2022

(30) Foreign Application Priority Data
Nov. 18, 2019 (JP) 2019-208015

(51) Int. Cl. G16H 50/20; A61B 5/35; A61B 5/352; A61B 5/00

(52) U.S. Cl. CPC G16H 50/20; A61B 5/35; A61B 5/352; A61B 5/7203

(57) ABSTRACT

The present invention provides a learning device including a learning unit that performs learning related to the output of vital data indicating life signs of a subject, with the use of first sensor data acquired from the subject by the first system as learning data and of teacher data based on second sensor data acquired from the subject by the second system in the same period as an acquisition period of the first sensor data, the second system being less affected by noises than the first system.

### TECHNICAL FIELD

The present invention relates to a learning device, a learning method, and a measurement device.

### BACKGROUND ART

Recently, there have been developed various kinds of devices that acquire vital data of subjects. For example, Patent Literature 1 discloses the technique of measuring an electrocardiographic waveform of a subject using electrodes provided at a seat and a steering of a mobile body. With such a technique, it is possible to reduce burdens on a subject that are caused by electrocardiographic waveform acquisition.

### CITATION LIST

### Patent Literature

Patent Literature 1: JP 2009-142575A

### SUMMARY OF INVENTION

### Technical Problem

However, in the technique described in Patent Literature 1, noises easily occur due to vibrations of a mobile body, the movement of a subject's body, and the like, which may reduce the acquisition accuracy of an electrocardiographic waveform.

Then, in view of the above-described problem, the present invention aims at providing a mechanism that is capable of acquiring vital data less affected by noises more efficiently.

### Solution to Problem

In order to solve the above-described problem, one aspect of the present invention provides a learning device including a learning unit that performs learning related to output of vital data indicating life signs of a subject, with the use of first sensor data acquired from the subject by a first system as learning data and of teacher data based on second sensor data acquired from the subject by a second system in a same period as an acquisition period of the first sensor data, the second system being less affected by noises than the first system.

In order to solve the above-described problem, another aspect of the present invention provides a learning method including performing learning related to output of vital data indicating life signs of a subject, with the use of first sensor data acquired from the subject by a first system as learning data and of teacher data based on second sensor data acquired from the subject by a second system in a same period as an acquisition period of the first sensor data, the second system being less affected by noises than the first system.

In order to solve the above-described problem, another aspect of the present invention provides a measurement device including a measurement unit that outputs vital data indicating life signs of a subject, with the use of first sensor data acquired from the subject by a first system as an input, in which the measurement unit outputs the vital data using a learned model constructed by learning related to output of the vital data with the use of the first sensor data as learning data and of teacher data based on second sensor data acquired from the subject by a second system in a same period as an acquisition period of the first sensor data, the second system being less affected by noises than the first system.

### Advantageous Effects of Invention

As described above, the present invention provides a mechanism that is capable of acquiring vital data less affected by noises more efficiently.

### BRIEF DESCRIPTION OF DRAWINGS

FIG. 1 is a diagram illustrating a functional configuration example of a learning device 10 according to an embodiment of the present invention.

FIG. 2 is a diagram illustrating a functional configuration example of a measurement device 20 according to the embodiment.

FIG. 3 is a diagram illustrating an example of a general electrocardiographic waveform in a single cycle.

FIG. 4 is a diagram illustrating examples of learning data and teacher data according to an embodiment of the present invention.

FIG. 5 is a diagram illustrating examples of an input and an output by a measurement unit 220 according to the embodiment.

FIG. 6 is a diagram illustrating examples of an input and an output by the measurement unit 220 according to the embodiment.

FIG. 7 is a flow chart illustrating a flow of a learning phase according to the embodiment.

FIG. 8 is a flow chart illustrating a flow of a measurement phase according to the embodiment.

### DESCRIPTION OF EMBODIMENTS

Hereinafter, referring to the appended drawings, preferred embodiments of the present invention will be described in detail. It should be noted that, in this specification and the drawings, structural elements that have substantially the same function and structure are denoted with the same reference signs, and repeated explanation thereof is omitted.

### Configuration Example

(Learning Device 10)

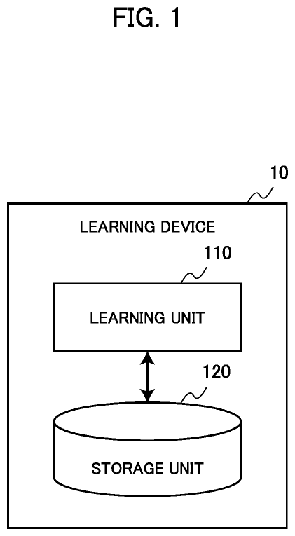

The learning device 10 of the embodiment may be a device that performs supervised learning with the use of, as an input, the same kind of sensor data acquired by two different systems in the same period. Here, the supervised learning indicates a method in which sets of input data (learning data) and correct answer data (teacher data) corresponding to the input data are provided to a computer so that the computer learns the correspondence therebetween. FIG. 1 is a diagram illustrating a functional configuration example of the learning device 10 according to the embodiment. As illustrated in FIG. 1, the learning device 10 of the embodiment may include a learning unit 110 and a storage unit 120. Note that the following will exemplify a case where the learning device 10 performs learning related to the output of vital data indicating life signs of a subject.

The learning unit 110 of the embodiment is characterized in performing learning related to the output of vital data with the use of the first sensor data acquired from a subject by the first system as learning data and of the second sensor data acquired from the subject by the second system in the same period as the acquisition time of the first sensor data, as teacher data, the second system being less affected by noises than the first system. In such a configuration, the correspondence relation between the first sensor data with many noises and the second sensor data less affected by noises is learned, whereby it is possible to generate a learned model that outputs vital data generated by removing noises from the first sensor data.

The learning unit 110 of the embodiment may perform the above-described learning using an arbitrary machine learning method capable of achieving supervised learning. The learning unit 110 performs learning using an algorithm such as a neutral network or a support vector machine (SVM), for example.

The functions of the learning unit 110 are achieved by a processor such as a graphics processing unit (GPU), for example. The details of the functions of the learning unit 110 according to the embodiment will be specifically described separately.

The storage unit 120 of the embodiment stores various kinds of information related to operations of the learning device 10. The storage unit 120 stores, for example, the first sensor data and the second sensor data, various kinds of parameters, and the like that are used in learning by the learning unit 110.

The above has described the functional configuration example of the learning device 10 according to the embodiment. Note that the configuration described above using FIG. 1 is merely an example and the configuration of the learning device 10 of the embodiment is not limited thereto. The learning device 10 of the embodiment may further include, for example, an operation unit that receives operations by an operator, an output unit that outputs various kinds of data, and the like. The configuration of the learning device 10 of the embodiment can be modified flexibly depending on specifications and uses.

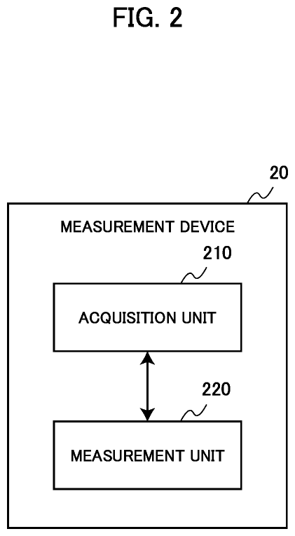

The following will describe a functional configuration example of the measurement device 20 according to the embodiment. The measurement device 20 of the embodiment may be a device that measures vital data using a learned model constructed by the learning device 10. FIG. 2 is a diagram illustrating a functional configuration example of the measurement device 20 according to the embodiment. As illustrated in FIG. 2, the measurement device 20 of the embodiment may include an acquisition unit 210 and the measurement unit 220.

The acquisition unit 210 of the embodiment is a component for acquiring the first sensor data from a subject. For this reason, the acquisition unit 210 of the embodiment includes various sensors in accordance with the characteristics of the first sensor data to be acquired.

The measurement unit 220 of the embodiment outputs vital data indicating life signs of a subject with the use of the first sensor data acquired by the acquisition unit 210 as an input. Here, the measurement unit 220 of the embodiment outputs vital data using a learned model constructed by learning by the learning unit 110. That is, the measurement unit 220 of the embodiment is characterized in outputting vital data using a learned model constructed by performing learning related to the output of vital data with the use of the first sensor data as learning data and of the teacher data based on the second data acquired from a subject by the second system in the same period as the acquisition period of the first sensor data, the second system being less affected by noises than the first system.

In the above-described configuration, it is possible to acquire, using only the first sensor data in which noises are assumedly mixed, high-accuracy vital data generated by removing the influence of such noises. Note that the functions of the measurement unit 220 of the embodiment are achieved by various processors.

The above has described the functional configuration example of the measurement device 20 according to the embodiment. Note that the configuration described above using FIG. 2 is a mere example and the functional configuration of the measurement device 20 of the embodiment is not limited thereto. The measurement device 20 of the embodiment may further include an operation unit, an output unit, an analysis unit that analyzes vital data, a notification unit that performs various notifications on the basis of analysis results, and the like. The configuration of the measurement device 20 of the embodiment may be modified flexibly in accordance with the characteristics of vital data to be measured, uses and utilizations of vital data, and the like.

<Details>

The following will describe the sensor data of the embodiment using concrete examples. Recently, there have been developed devices that acquire various kinds of sensor data. Moreover, even in the case of acquiring the same kind of sensor data, a plurality of systems may exist. Here, it is assumed that the change in voltage caused by the cardiac activity of a subject is acquired as an electrocardiographic waveform.

The system of acquiring an electrocardiographic waveform may be, for example, a system of a 12 inductive electrocardiogram in which a plurality of electrodes are attached directly on the skin of a subject so that the change in voltage is recorded with the electrodes. With such a system, it is possible to acquire a high-accuracy electrocardiographic waveform less affected by noises. Meanwhile, such a system may often limit activities of a subject, or may cause a subject to feel annoyed because the electrodes are attached directly onto the skin.

Moreover, another system for acquiring an electrocardiographic waveform may be a system in which with electrodes provided at a plurality of positions to be assumedly in contact with a subject, a change in voltage acquired when the subject comes into contact with the electrodes is recorded. Such a system is used to acquire an electrocardiographic waveform of a subject operating a device, for example. As an example, there is known a technique of acquiring an electrocardiogram of a driver driving a mobile body such as a vehicle using electrodes provided at a steering or a driver's seat with which the driver assumedly comes into contact during driving. With such a technique, it is not necessary to attach electrodes directly onto the skin of the driver, whereby an electrocardiographic waveform can be acquired without requiring driver's consciousness. In such a case, meanwhile, noises easily occur due to the movement of a driver's body caused by driving action, vibrations of a vehicle, and the like, which may deteriorate the accuracy of an acquired electrocardiographic waveform.

As described above, each of a plurality of systems for acquiring sensor data has an advantage, while there may exist a case where the accuracy of acquired sensor data varies. Therefore, there has been demanded a technique of improving the acquisition accuracy of sensor data while making use of the advantage of a certain system.

To solve the above-described aspect, the learning unit 110 of the embodiment performs learning with the use of first sensor data acquired by the first system as learning data and of teacher data based on the second sensor data acquired by the second system in the same period as the acquisition period of the first sensor data, the second system being less affected by noises than the first system. In this manner, it is possible to construct a learned model that outputs high-accuracy vital data less affected by noises, even from only the first sensor data.

The following will exemplify a case where the vital data of the embodiment is data related to the cardiac activity. In this case, the learning unit 110 may learn the output of data related to the inspected cardiac activity of a subject with the use of the first electrocardiographic waveform acquired by the first system as learning data and of teacher data based on the second electrocardiographic waveform acquired by the second system in the same period as the acquisition period of the first electrocardiographic waveform.

In this case, the above-described first system may be a system of acquiring an electrocardiographic waveform using at least two electrodes to be assumedly in contact with a subject, and the above-described second system may be a system of acquiring an electrocardiographic waveform using at least two electrodes attached directly on the skin of the subject.

For example, when the subject is a driver driving a mobile body such as a vehicle, two electrodes used in the above-described first system may be provided at a seat on which the subject is seated and at a device operated by the subject (a steering, for example).

In the above-described configuration, it is possible to acquire high-accuracy data generated by removing noises occurred due to the movement of a driver's body, vibrations of the vehicle, and the like, while keeping the advantages of the first system such as that the driver is not caused to feel annoyed.

Note that the learning unit 110 of the embodiment may perform learning related to the output of the third electrocardiographic waveform generated by removing noises from the first electrocardiographic waveform, with the use of the second electrocardiographic waveform itself as teacher data. In this case, it is possible to acquire various physiological indices by analyzing the third electrocardiographic waveform in accordance with objects.

Meanwhile, in a case where a physiological index to be acquired from an electrocardiographic waveform is preliminarily determined, it is also possible to cause the learning unit 110 to learn specific feature points in accordance with such a physiological index. Here, the feature points (feature waveform) of a general electrocardiographic waveform will be described.

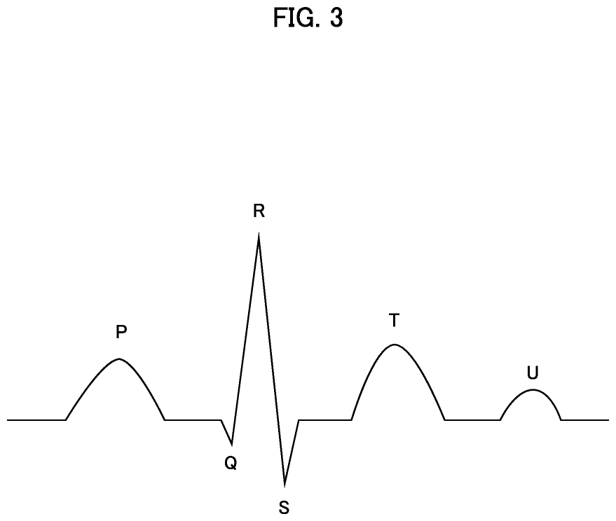

FIG. 3 is a diagram illustrating an example of a general electrocardiographic waveform in a single cycle. Note that in FIG. 3, the horizontal axis indicates the lapse of time, and the vertical axis indicates a change in voltage. As illustrated in FIG. 3, a plurality of feature waveforms exhibiting characteristic forms can be observed in the general electrocardiographic waveform. The examples of the feature waveform include a P wave, Q wave, R wave, S wave, QRS wave (formed by a Q wave, R wave, and S wave), T wave, U wave, and the like.

Among them, the R wave, for example, is an important feature waveform as an index of heartbeat variation (fluctuation). The interval between an R wave in a cycle and an R wave in the following cycle (RRI: R-R Interval) is used to calculate a heartbeat cycle. It is also known that a fluctuation occurs in the RRI due to stress and tiredness, and thus the RRI is an effective physiological index also for detecting a physical burden or mental burden of a subject. In addition, the Q-T interval (QTI) between a Q wave and a T wave in a cycle, for example, indicates time from the start of ventricular excitation to the disappearance of the excitation, and is an important physiological index for detecting an irregular pulse or the like.

From this, with the use of presence probability data that indicates the presence probability of a feature point in the second electrocardiographic waveform and is acquired from the second electrocardiographic waveform, as teacher data, the learning unit 110 of the embodiment may perform learning related to the output of presence probability data indicating the presence probability of the above-described specific feature point in the first electrocardiographic waveform.

The learning unit 110 of the embodiment may perform learning related to the output of presence probability data indicating the presence probability of an R wave in the first electrocardiographic waveform, with the use of presence probability data indicating the presence probability of an R wave in the second electrocardiographic waveform as teacher data, for example.

In the above-described learning, it is possible to construct a learned model that detects an arbitrary feature point such as an R wave, for example, with high accuracy. Moreover, with this learned model, a physiological index such as the RRI of a subject can be measured in real time.

In this manner, the leaning unit 110 of the embodiment may perform learning using the teacher data in accordance with the usage application of the measurement device 20 in which the learned model is provided.

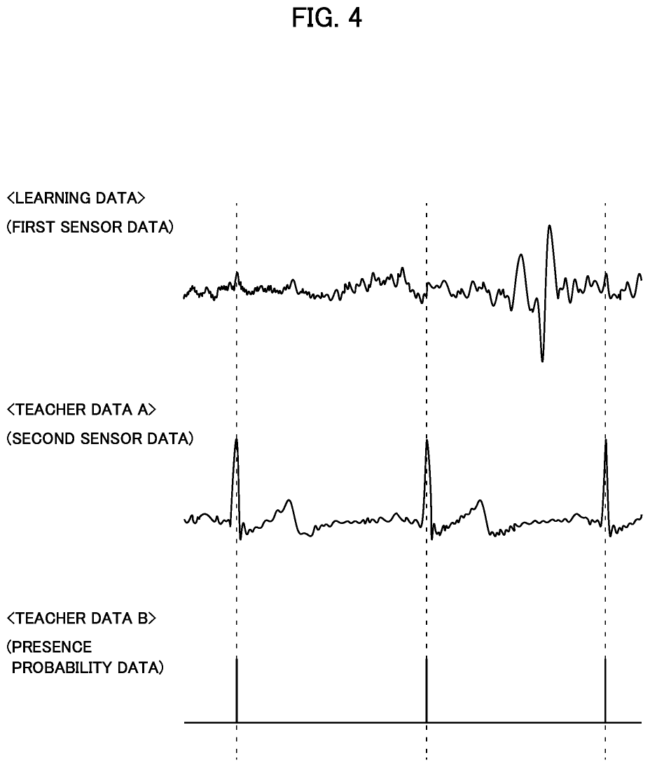

FIG. 4 is a diagram illustrating examples of learning data and teacher data according to the embodiment. FIG. 4 illustrates, in the upper stage thereof, the first sensor data (first electrocardiographic waveform) used as learning data. FIG. 4 also illustrates, in the middle stage thereof, the second sensor data (second electrocardiographic waveform) acquired in the same period as the acquisition period of the first sensor data and used as teacher data A. FIG. 4 also illustrates, in the lower stage thereof, presence probability data of an R wave generated on the basis of the above-described second sensor data and used as teacher data B. Note that in FIG. 4, the position of an R wave (R wave peak) in each data is illustrated by a dotted line.

As illustrated in FIG. 4, the first sensor data acquired by the first system includes many noises, and with the first sensor data as it is, detection of an R wave may not be performed preferably. Here, with the use of the second sensor data less affected by noises as the teacher data A, it is possible to allow the learning unit 110 to learn the correspondence relation between the first sensor data and the second data.

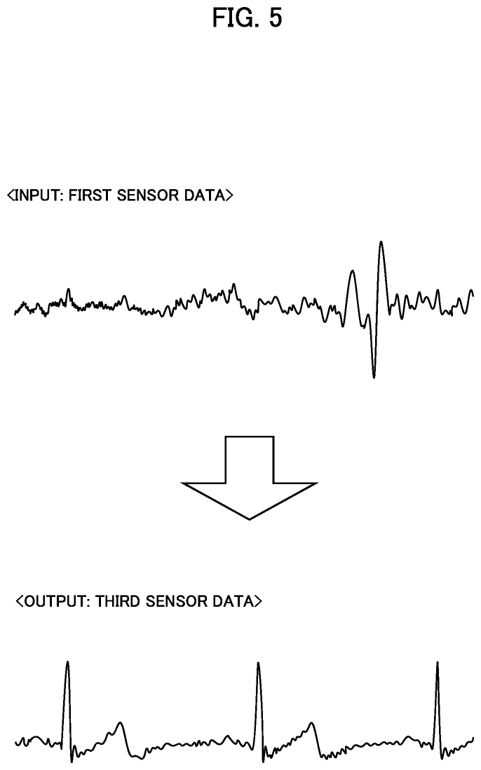

Using the learned model constructed by the above-described learning, the measurement unit 220 of the embodiment can output the third sensor data (third electrocardiographic waveform) generated by removing noises from the first sensor data as an input, as illustrated in FIG. 5. In this manner, it is possible to acquire various physiological indices related to a subject with high accuracy by performing arbitrary processing and analysis on the output third sensor data.

Meanwhile, with the use of the presence probability data illustrated in FIG. 4 as the teacher data B, it is possible to allow the learning unit 110 to directly learn the correspondence relation between the first sensor data and an arbitrary feature point.

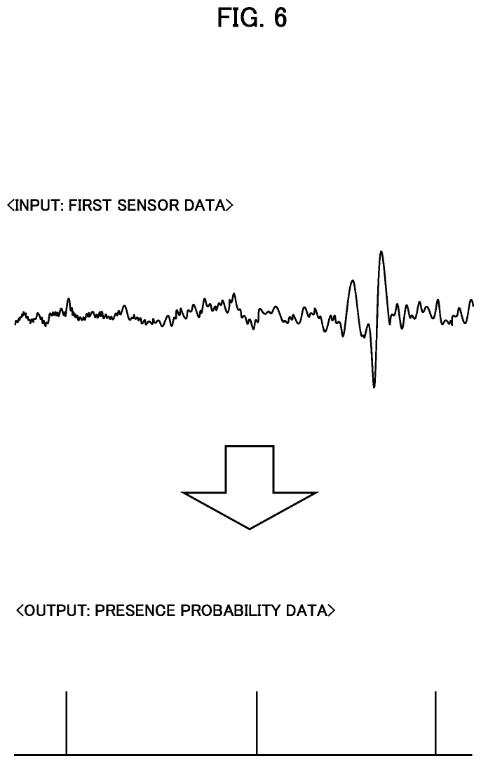

In this case, the measurement unit 220 of the embodiment can output the presence probability data related to a specific feature point such as an R wave, for example, with the first sensor data as an input, as illustrated in FIG. 6. In this manner, it is possible, for example, to measure a physiological index such as an RRI in real time and perform various actions in accordance with the measurement value. Although FIG. 4 and FIG. 5 exemplify the case in which the presence probability data is of two values of 0 (absent) or 1 (present), the presence probability data of the embodiment may be of three or more values.

<Flow of Learning Phase and Measurement Phase>

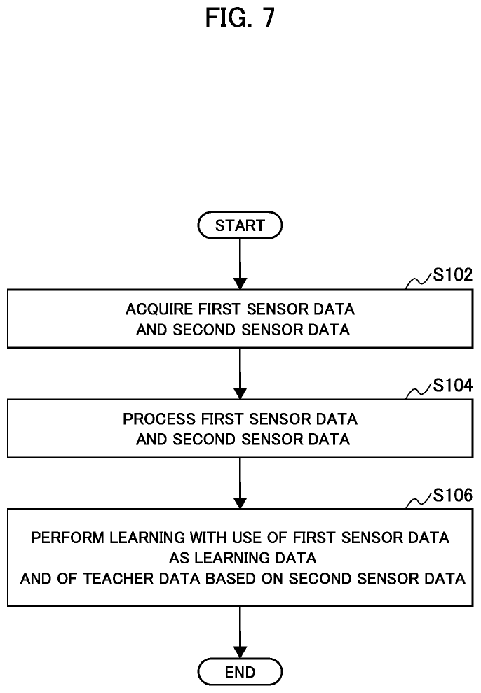

The following will describe flows of the learning phase for learning using the learning device 10 and the measurement phase for measurement using the measurement device 20 according to the embodiment. FIG. 7 is a flowchart illustrating a flow of the learning phase according to the embodiment.

As illustrated in FIG. 7, in the learning phase of the embodiment, the first sensor data and the second sensor data are acquired first (S102). Here, the first sensor data and the second sensor data may be acquired together with the information of time stamps and the like so that the synchronization in the time axis is possible. Moreover, the first sensor data and the second sensor data may be acquired by a separate device from the learning device 10. The acquired first sensor data and second sensor data are stored in the storage unit 120 of the learning device 10.

Next, the first sensor data and the second sensor data are processed if necessary (S104). For example, in a case where the presence probability data related to a specific feature point is used as teacher data, the processing of converting the second sensor data acquired at Step S102 into presence probability data may be performed at Step S104. Moreover, various kinds of filter processing for reducing noises in the first sensor data and the second sensor data, or the like may be performed. Note that the above-described processing may be performed by a separate device from the learning device 10.

Next, the learning unit 110 performs learning with the use of the first sensor data as learning data and of the teacher data based on the second sensor data (S106). Here, the learning unit 110 may use the second sensor data itself (or the second sensor data having been subjected to filter processing) as teacher data, or the presence probability data generated at Step S104 as teacher data.

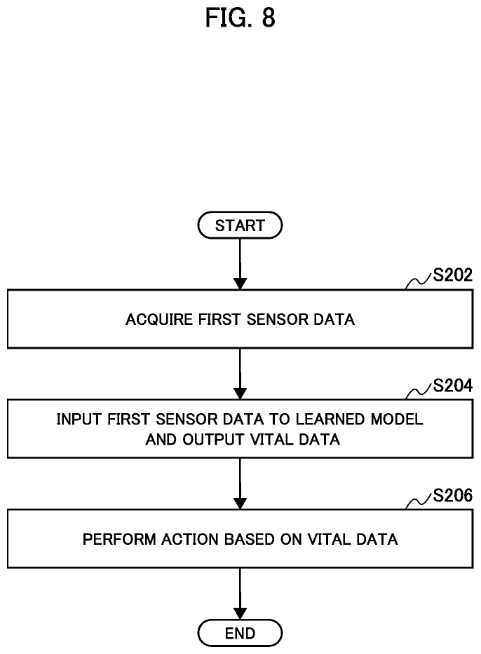

The above has described the flow of the learning phase according to the embodiment. The following will describe a flow of the measurement phase according to the embodiment. FIG. 8 is a flow chart illustrating a flow of the measurement phase according to the embodiment.

As illustrated in FIG. 8, in the measurement phase of the embodiment, the acquisition unit 210 first acquires the first sensor data by the first system (S202). The acquisition unit 210 may acquire, as the first sensor data, an electrocardiographic waveform of a driver using a plurality of electrodes arranged at a steering and a seat of a vehicle, for example.

Next, the measurement unit 220 inputs the first sensor data acquired at Step S202 to a learned model and outputs vital data (S204). In a case where the learning is performed with the use of the second sensor data as teacher data in the learning phase, the above-described vital data may be the third sensor data generated by removing noises from the first sensor data. Meanwhile, in a case where the learning is performed with the use of the presence probability data as teacher data in the learning phase, the above-described vital data may be presence probability data indicating the presence probability of an arbitrary feature point.

Next, various actions based on the vital data output at Step S204 are performed if necessary (S206). The above-described action may be, for example, a notification or the like based on an RRI detected from the vital data. The above-described action may be performed by a separate device from the measurement device 20.

<Supplement>

Heretofore, preferred embodiments of the present invention have been described in detail with reference to the appended drawings, but the present invention is not limited thereto. It is obvious that a person skilled in the art can arrive at various alterations and modifications within the scope of the technical ideas defined in the claims, and it should be naturally understood that such alterations and modifications are also encompassed by the technical scope of the present invention.

For example, the above-described embodiment has described, as a main example, the case in which the learning unit 110 performs learning related to the output of vital data indicating life signs of a subject. However, the object to be learned by the learning unit 110 is not limited to the output of vital data. The learning unit 110 may perform learning related to the output of data or the like indicating an operating state of an arbitrary device, for example.

Moreover, the above-described embodiment has exemplified, as the first system of acquiring an electrocardiographic waveform, the system in which electrodes are arranged at positions to be assumedly in contact with a subject, and has exemplified, as the second system, the system in which electrodes are attached directly on the skin of a subject. However, the first system and the second system in the present technique may be arbitrary different systems having a difference in susceptibility to influences by noises. For example, in the case of acquiring a heartbeat, the first system may be a non-contact system using a doppler sensor, and the second system may be a contact system with electrodes attached on the skin of a subject.

A sequence of processing by the devices described in this specification may be achieved using any one of software, hardware, and the combination of software and hardware. A program forming the software is preliminarily stored in, for example, a recording medium (non-transitory media) provided inside or outside each device. Then, each program is read in a random access memory (RAM) when executed by a computer, and executed by a processor such as a central processing unit (CPU). The above-described recording medium is, for example, a magnetic disk, an optical disk, a magneto-optical disk, a flash memory, or the like. Moreover, the above-described computer program may be distributed through a network, for example, without using any recording medium.

### REFERENCE SIGNS LIST

10 learning device
110 learning unit
120 storage unit
20 measurement device
210 acquisition unit
220 measurement unit

1. A learning device, comprising:
a learning unit that performs learning related to output of vital data indicating life signs of a subject, with the use of first sensor data acquired from the subject by a first system as learning data and
of teacher data based on second sensor data acquired from the subject by a second system in a same period as an acquisition period of the first sensor data, the second system being less affected by noises than the first system.

2. The learning device according to claim 1, wherein
the vital data includes data related to a cardiac activity, and
the learning unit learns output of data related to the cardiac activity of the subject, with the use of a first electrocardiographic waveform acquired by the first system as learning data and of teacher data based on a second electrocardiographic waveform acquired by the second system in a same period as an acquisition period of the first electrocardiographic waveform.

3. The learning device according to claim 2, wherein the learning unit performs learning related to output of a third electrocardiographic waveform generated by removing noises from the first electrocardiographic waveform with the use of the second electrocardiographic waveform as teacher data.

4. The learning device according to claim 2, wherein the learning unit performs learning related to output of presence probability data indicating a presence probability of a specific feature point with the use of, as teacher data, presence probability data that indicates the presence probability of the specific feature point in the second electrocardiographic waveform and is acquired from the second electrocardiographic waveform.

5. The learning device according to claim 4, wherein
the specific feature point includes an R wave in an electrocardiographic waveform, and
the learning unit performs learning related to output of presence probability data indicating a presence probability of the R wave in the first electrocardiographic waveform.

6. The learning device according to claim 2, wherein
the first system is a system for acquiring an electrocardiographic waveform using at least two electrodes to be assumedly in contact with the subject, and
the second system is a system for acquiring an electrocardiographic waveform using at least two electrodes attached on a skin of the subject.

7. The learning device according to claim 6, wherein the two electrodes used in the first system are provided at a seat on which the subject is seated and at a device to be operated by the subject.

8. The learning device according to claim 1, wherein the subject is a driver driving a mobile body.

9. A learning method, comprising:
performing learning related to output of vital data indicating life signs of a subject, with the use of first sensor data acquired from the subject by a first system as learning data and
of teacher data based on second sensor data acquired from the subject by a second system in a same period as an acquisition period of the first sensor data, the second system being less affected by noises than the first system.

10. A measurement device, comprising:
a measurement unit that outputs vital data indicating life signs of a subject, with the use of first sensor data acquired from the subject by a first system as an input, wherein
the measurement unit outputs the vital data using a learned model constructed by learning related to output of the vital data with the use of the first sensor data as learning data and of teacher data based on second sensor data acquired from the subject by a second system in a same period as an acquisition period of the first sensor data, the second system being less affected by noises than the first system.
